(19) United States
(12) Patent Application Publication
(10) Pub. No.: US 2007/0135769 A1
(43) Pub. Date: Jun. 14, 2007

(54) Device and method for supporting a medical device

(76) Inventor: Dennis R. Tollini, Clarence Center, NY (US)

Correspondence Address: SIMPSON & SIMPSON, PLLC, 5555 MAIN STREET, WILLIAMSVILLE, NY, 14221-5406, US

(21) Appl. No.: 11/298,806

(22) Filed: Dec. 9, 2005

(51) Int. Cl. A61M 5/32

(52) U.S. Cl. 604/174

(57) ABSTRACT

The invention broadly comprises a device for controlling a cannulation angle for a fistula needle including a pre-formed support element or a piece of material layered to form a thickness. The element or material supports the needle and is manipulatable to control the angle. In some aspects, the element is resilient and arranged to control the angle in response to pressure upon the support element. In some aspects, the support element is configured to holdingly engage the fistula needle. In some aspects the angle is with respect to a surface and the support element is movable along the surface. The invention also broadly comprises a combination including a pre-formed support element and a fistula needle. The support element is arranged to support the fistula needle and to move with respect to the fistula needle. The angle for the fistula needle is controllable via the movement of the support element.

### FIELD OF THE INVENTION

The invention relates generally to providing controllable support for medical devices. In particular, the invention relates to a device for controlling an angle of entry for a medical device. Specifically, the invention controls the angle of cannulation for a fistula needle in response to pressure applied to the device or to movement of the device.

### BACKGROUND OF THE INVENTION

Fistula needles are used for the purpose of accessing a patient's vascular system. For example, for patients requiring dialysis, a fistula or graft can be surgically placed between a vein and an artery to create a “pumping station” and to avoid the necessity of directly accessing an artery. A fistula needle is used to access the fistula or graft. In general, the fistula needle must be maintained at a certain angle of cannulation to ensure proper results. That is, different patients achieve acceptable flow rates at different angles of cannulation. Also, the position of the tip of the needle in the fistula or graft may be important, i.e., a minimal length of the needle may be required within the fistula or graft. The optimal angle of cannulation to achieve the acceptable flow rate or length of needle may not be readily apparent and may change over time for the same patient. For example, the body naturally creates a layer of scar tissue over a fistula. In time this layer of tissue increases in thickness and or surface extent. Therefore, to reach the fistula, different angles of cannulation may be required depending on the configuration of the layer of tissue. The orientation of the fistula needle within the fistula also is important. For example, if the open end of the fistula needle presses against the side of the fistula, the flow through the needle is undesirably reduced or even stopped.

Unfortunately, it is presently difficult to support a fistula needle in stable positions during the process of finding an optimal angle of cannulation and to then maintain the fistula needle at the desired angle. For example, it is known to use a folded or crumpled gauze pad as a support for a fistula needle. An attempt to control the angle of cannulation can be made by varying the pressure applied by the fistula needle on the pad. Unfortunately, a folded or crumpled gauze pad does not have sufficient “compression memory.” That is, the pad may not maintain a fistula needle at a set angle in response to a set pressure of the fistula needle on the pad. Typically, the pad is initially too soft and allows the fistula needle to compress the pad further than is necessary to maintain a desired angle and then becomes too compressed to allow the fistula needle to move to the desired angle. Also, a folded pad lack sufficient uniformity in its compressibility. That is, the pad will tend to initially compress too easily and not be able to maintain a fistula needle at a desired position up to a point of not be compressible enough. Further, the pad does not maintain its shape unless it is fixed with tape or constrained in some other manner, which may not be particularly effective and which greatly complicates the overall cannulation process.

Some fistula needles include a relatively long and rigid body that extends above the body of the patient. Unfortunately, it is difficult at present to properly secure such needles and these needles tend to “teeter totter” at a point along the length of the rigid body, which causes the end of the needle to undesirably move within the fistula or graft. This movement can cause the open end of the fistula needle to press against the side of the fistula, undesirably reducing or even stopping the flow through the needle.

Economic considerations are important for fistula needle applications, such as dialysis, which are repeated at frequent intervals for extended periods of time for a same patient. Also of note is the large number of patients requiring fistula needle applications such as dialysis. Therefore, due to the general desirability of reducing costs and the specific cost reduction pressures associated with current health care management and distribution systems, fistula needle support means should be relatively inexpensive to manufacture, distribute, store, and use, as well as provide a simple means of finding angles to provide optimal flow rates. Ease of use also is an important factor given the frequency and ubiquitous nature of many fistula needle applications, such as dialysis. Therefore, fistula needle support means should be as compact, light-weight, and versatile as possible. For some applications, modern medical practice relics on the use of disposable devices that are pre-sterilized and/or the sterility of which can be easily maintained. Therefore, fistula needle support means should optionally be able to meet the preceding criteria.

U.S. Pat. No. 5,911,707 (Wolvek et al.) discloses a needle guide for an angiographic needle. In general, a predetermined angle is desirable for angiographic applications and Wolvek teaches a guide with support members 30 and 32 at set angles. Unfortunately, the angle provided by the guides is not variable and the application addressed by Wolvek is not relevant to fistula needles and dialysis applications. Wolvek also teaches a support surface for the support members, which is unsuited for many applications, such as, cannulation of fistulas. Unfortunately, Wolvek's device does not appear to be cost-effective or easy to use for applications such as dialysis and may be problematic regarding disposability. Also, sterility, if desired or necessary, would be problematic.

U.S. Pat. No. 3,288,137 (Lund) discloses an anchoring assembly for an infusion needle. In general, Lund is solving the problem of holding an infusion needle, not a fistula needle. Further, Lund is solving the problem of holding the infusion needle in place and preventing the needle from being dislodged. Lund's device is quite clumsy and not designed or well-suited for finding or controlling an optimal angle of cannulation for a fistula needle. Lund discloses an assembly including a number of components including a part made of a deformable material, such as wire mesh, attached to a pads and bases fixed to a patient's body. The deformable material can be bent to vary an angle of the infusion needle. Unfortunately, Lund's assembly, which consists of at least three different components, appears to be very elaborately constructed, costly, and inapplicable for applications such as dialysis. The device also appears to be awkward to use. Sterility, if desired or necessary, would be problematic.

U.S. Pat. No. 6,827,705 (Bierman) discloses a catheter anchoring system. The patent teaches a support member for holding a catheter. Bierman is not addressing fistula needles and applications such as dialysis and Bierman's device would be overly elaborate and costly for such applications. Bierman owns a number of patents and patent applications disclosing a similar catheter anchoring system, for example, U.S. Pat. Nos. 6,786,892 and 5,456,671. For the sake of brevity, these documents are not further discussed.

U.S. Pat. No. 4,397,641 (Jacobs) also teaches a device for anchoring a catheter. Unfortunately, Jacob does not vary the angle of the catheter using his device and teaches holding a catheter at a fixed angle in a fixed location. Also, Jacobs is not addressing fistula needles and applications such as dialysis and Jacob's device would be overly elaborate and costly for such applications.

A number of U.S. Pat. Nos., 5,662,617; 5,024,665; 4,883,053; 4,711,636; and 2,402,306 (Odell et al., Kaufman, Simon, Bierman, and Turkel, respectively) teach a “mechanical” means for varying the angle of a catheter and anchoring a catheter. These patents do not addressing fistula needles and applications such as dialysis These mechanical means include machined components such as base plates, swivels, locking mechanisms, slides, knurls, ratchets, etc. Unfortunately, these devices appear to be very elaborately constructed and would be costly for applications such as dialysis. The devices also appear to be awkward to use. Sterility, if desired or necessary, would be problematic.

Thus, there is a long-felt need to provide a cost-effective and easy to use means for controlling an angle of cannulation for a fistula needle in accordance with current medical practice.

### SUMMARY OF THE INVENTION

The invention broadly comprises a device for controlling an angle of cannulation for a fistula needle, including a pre-formed support element operatively arranged to support the fistula needle and manipulatable to control the angle of cannulation. In some aspects, the support element is resilient and the support element is arranged to control the angle in response to pressure upon the support element. In some aspects, the support element has a solid shape or includes at least one void. The support element has a cross-section with an outline and the outline is selected from the group consisting of round, ovoid, arcuate, three-sided, four-sided, having more than four sides, having straight edges and arcuate edges, and amorphous.

In some aspects, the support element is a pouch at least partially filled with a material selected from the group consisting of one or more first gases; a solid mass; a plurality of pieces of a first solid material; a first liquid; and a combination of one or more second gases, or a second liquid, or at least one piece of a second solid material.

In some aspects, the support element is a coil. In some aspects, the support element is configured to holdingly engage the fistula needle and includes a slit or cut-out arranged to receive the fistula needle. In some aspects, the angle is with respect to a surface and the support element is movable along the surface.

The invention also broadly comprises a device for controlling an angle of cannulation for a fistula needle including a piece of material layered to form a thickness and operatively arranged to support the fistula needle. At least portions of the material are detachably connectable to each other and the angle is controllable via the thickness. In some aspects, the piece of material is folded or rolled. In some aspects, the layered material is resilient and the layered material is arranged to control the angle in response to pressure upon the layered material. In some aspects, the angle is with respect to a surface and the support element is operatively arranged to move along the surface and to control the angle in response to the movement.

The invention further broadly comprises a combination for controlling an angle of cannulation, including a pre-formed support element and a fistula needle. The support element is arranged to support the fistula needle and to move with respect to the fistula needle. An angle of cannulation for the fistula needle is controllable via the movement of the support element. In some aspects, the support element is resilient and the support element is arranged to control the angle is response to pressure upon the support element. In some aspects, the support element is a pouch or a piece of material layered to form a thickness. At least portions of the material are detachably connectable to each other and the angle is controllable via the thickness. In some aspects, the piece of material is folded or rolled. In some aspects, the support element comprises a coil or the support element is configured to holdingly engage the fistula needle.

The invention also broadly comprises a method for controlling an angle of cannulation for a fistula needle.

It is a general object of the present invention to provide a simple and easy-to-use device and method for controlling an angle of cannulation for a fistula needle.

It is another object of the present invention to provide a device and method for controlling an angle of cannulation using simple, cost-effective materials.

It is still another object of the present invention to provide a device and method for controlling an angle of cannulation that is easy for medical practioners to use and is compatible with medical protocols.

These and other objects and advantages of the present invention will be readily appreciable from the following description of preferred embodiments of the invention and from the accompanying drawings and claims.

### BRIEF DESCRIPTION OF THE DRAWINGS

FIG. 1 is a perspective view of a present invention device for controlling an angle of cannulation for a fistula needle;

FIGS. 2A and 2B are perspective views of a present invention device showing varying degrees of compression for a support element;

FIG. 3 is a side view of a present invention device with a tubular support element;

FIGS. 4A and 4B are side views of respective present invention devices showing a difference in height between support elements for the devices;

FIG. 5A is a perspective view of a present invention support element with a slit;

FIG. 5B is a perspective view of a present invention support element with a cut-out;

FIG. 5C is a partial cross-sectional view of a present invention device showing a slit or a cut-out;

FIGS. 6A and 6B are perspective views of a present invention device with a support element movable with respect to a fistula needle;

FIG. 7 is a perspective view of a present invention device with a pouch support element;

FIGS. 8A, 8B, and 8C are side views of a present invention device with a layered support element;

FIG. 9 is a perspective view of a present invention device with a coil support element; and,

FIG. 10 is a front view of a present invention device with a support element having a shaped bottom surface.

### DETAILED DESCRIPTION OF THE PREFERRED EMBODIMENT

At the outset, it should be appreciated that like drawing numbers on different drawing views identify identical, or functionally similar, structural elements of the invention. While the present invention is described with respect to what is presently considered to be the preferred aspects, it is to be understood that the invention as claimed is not limited to the disclosed aspects.

Furthermore, it is understood that this invention is not limited to the particular methodology, materials and modifications described and as such may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. Although any methods, devices or materials similar or equivalent to those described herein can be used in the practice or testing of the invention, the preferred methods, devices, and materials are now described.

FIG. 1 is a perspective view of present invention device 10 for controlling an angle of cannulation for a fistula needle. It should be understood that the present invention is not limited to the following examples and applications. The present invention can be used in a large variety of applications and use in such variety of applications is within the spirit and scope of the invention as claimed. FIG. 1 shows a typical example of a present invention device in use. For example, tape 11 has been applied to support element 12 and fistula needle assembly 14 to hold the support element and needle in place with respect to a patient's arm 16. Specifically, needle assembly 14 is secured at a desired angle 18 formed between the needle and the arm. For the sake of clarity and clear visualization, angle 18 is shown with respect to different portions of needle assembly 14 in the figures described below. However, it should be understood that angle 18 is with respect to the angle of the needle assembly with respect to the patient's body. In FIG. 1, a fistula or graft (not shown) has been surgically created or placed, respectively, between a vein (not shown) and an artery (not shown) in arm 16 to create a “pumping station.”

In general, specific or optimal angles 18 are needed to ensure proper results. For example, different patients achieve acceptable flow rates at different angles of cannulation (also referred to as cannulation angles). As noted supra, the optimal angle of cannulation is dependent at least upon the configuration of tissue over the fistula and desired orientation of the fistula needle within the fistula. In general, the type of attachment shown in FIG. 1 is applicable to the present invention devices described below. However, it should be understood that any method known in the art can be used to secure the present invention and that such methods are included in the spirit and scope of the invention as claimed.

In FIG. 1, element 12 is used to support fistula needle assembly 14, shown in use with a patient's arm 16. However, it should be understood that device 10 is not limited to use with a fistula needle assembly or with a patient's arm. Device 10 can be used with other medical devices and on other parts of a patients body and such uses are within the spirit and scope of the invention as claimed. Specifically, device 10 can be used with any medical device for which control of an angle of entry is desirable or necessary. During the process of finding a suitable angle 18, element 12 provides stable support for the fistula needle assembly. Element 12 maintains the angle of cannulation 18 for the fistula needle with respect to arm 16 and is manipulatable to control angle 18. In some aspects, element 12 includes axis 19 as is further described infra. It should be understood that the discussion regarding FIG. 1 may be applicable to all aspects of the present invention.

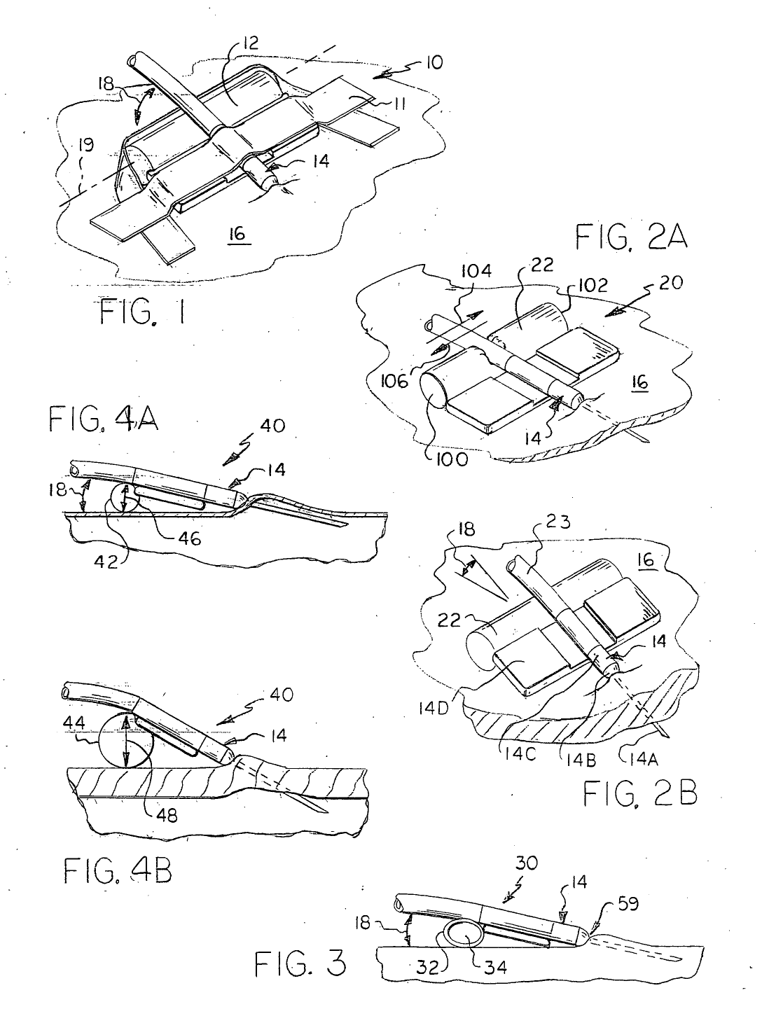

FIGS. 2A and 2B are perspective views of present invention device 20 showing varying degrees of compression for support element 22. As shown in FIG. 2B, needle assembly 14 includes needle 14A, hub 14B, connector/body 14C, and wing 14D. Tubing 23 is connected to body 14C. Hereinafter, needle assembly 14 is referred to as needle 14. It should be understood that the present invention is not limited to use with the fistula needle assembly configuration shown in the drawings and that use with other types and configurations of fistula needles is within the spirit and scope of the invention as claimed. Alternately stated, the present invention can be used with any fistula needle assembly known in the art. Device 20 includes pre-formed support element 22. By pre-formed, we mean that the element is used in substantially the shape in which the element is received by a user of the device and the shape of the element is not substantially altered during use. For example, the element is not wadded or crumpled, for example, as might be done with a gauze pad. As noted infra, some change in the shape of the element may occur as a result of pressure applied to the element, but the essential shape of the element is not altered.

In some aspects, element 22 is resilient and angle 18 is controllable in response to pressure applied to element 18, for example, by pushing on needle 14. For example, in response to a substantially constant pressure from fistula needle 14 (for example, as applied by a practioner to the fistula needle) element 22 reaches a stable position and then maintains a substantially constant angle 18. That is, the practioner does not have to ‘hold-up’ fistula needle 14 to maintain angle 18. Element 22 has sufficient resiliency and “compression memory” to prevent fistula needle 14 from continuing to ‘sink’ into element 22 once the stable position is reached. Element 22 ‘pushes back’ to a certain degree in response to pressure applied to element 22, for example, by fistula needle 14. The ‘push back’ on the fistula needle also can be a useful counterpoint for use by a practioner in determining an optimal angle of cannulation. For example, a practioner can push the fistula needle against element 22 and then controllably allow the ‘push back’ to move the fistula needle to a desired position. Once the desired position is reached, the practioner can stabilize fistula needle 14 by taping or otherwise securing the fistula needle to a patient's body as shown in FIG. 1. In this case, the resiliency of element 22 and the force applied by the tape oppose each other and help maintain the stability of the fistula needle and angle 18.

In response to a variation of the pressure of fistula needle 14 on element 22, angle 18 is variable. For example, in FIG. 2A, more pressure is applied to needle 14 than in FIG. 2B. Therefore, element 22 compresses more in FIG. 2A, resulting in a smaller angle 18 than in FIG. 2B. In both figures, the support element is arranged to maintain fistula needle 14 at a specific angle in response to a specific pressure on fistula needle 14. Alternately stated, element 22 does not continue to give way once the stable position noted supra is achieved.

In some aspects and as shown in FIGS. 2A and 2B, element 22 is a solid shape. By solid shape we mean that element 22 is substantially free of voids or is formed of a solid material.

FIG. 3 is a side view of present invention device 30 with tubular support element 32. Device 30 includes support element 32 having a void or voids. For example, the element is a tube or has a pipe shape, as shown in FIG. 3. The void or voids may be enclosed within element 32 or may be open to the exterior of element 32, for example, opening 34. In some aspects, in response to varying the pressure on needle 14, element 32 changes shape and thus, angle 18 changes. For example, as the pressure is increased, element 32 “flattens.” The tubing or tubular element used for element 32 can be selected to control the compressibility of element 32. For example, tubing can be soft (more compressible) or hard (less compressible). Element 32 can be made of any type of tubing or tubular elements known in the art. In particular, element 32 can be made of medical grade tubing, which could be made of silicon, polycarbonate or any other material known in the art. The positioning of the needle with respect to element 32 also can affect the compressibility of the element. For example, a segment of tubing making up element 32 may be less compressible in the middle (longitudinally) than at the ends.

FIGS. 4A and 4B are side views of respective present invention devices showing a difference in height between support elements 42 and 44, respectively. In some aspects, the height of the support element, with respect to the surface supporting the element, is varied to control angle 18. For example, in FIG. 4A, height 46 of support element 42 is less than height 48 of support element 44. Therefore, angle 18 is less in FIG. 4A than in FIG. 4B.

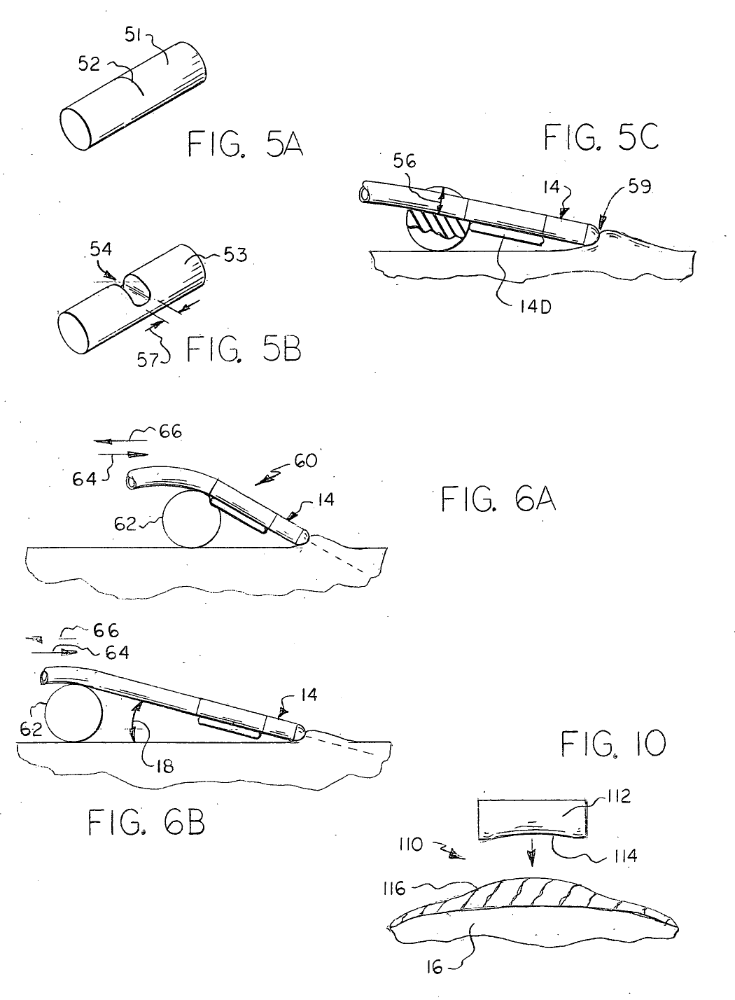

FIG. 5A is a perspective view of support element 51 with slit 52.

FIG. 5B is a perspective view of support element 53 with cut-out 54.

FIG. 5C is a partial cross-sectional view of device 50 showing element 51 or element 53. The following should be viewed in light of FIGS. 5A-5C. Support elements 51 and 53 are configured to holdingly engage fistula needle 14. By holdingly engage we mean that the elements are configured to receive the fistula needle and hold the fistula needle to some degree. In FIG. 5A, support element 51 has slit 52 for holding needle 14. Slit 52 engages needle 14 and, in some cases, in conjunction with wings 55 of needle 14, holds needle 14 in place. Slit 52 is not limited to any particular depth 56.

In FIG. 5B, support element 53 includes cut-out 54 that engages needle 14 and, in some cases, in conjunction with wings 55, holds needle 14 in place. Cut-out 54 is shown with a semi-circular shape. However, it should be understood that cut-out 54 is not restricted to any particular shape. Cut-out 54 is not limited to any particular width 57 or depth 56 or any particular ratio of width and depth. Cut-out 54 can be sized so that the fistula needle fits snuggly in the cut-out or the cut-out can be sized so that the fistula needle fits more loosely in the cut-out.

In general, wings 55 rest on element 51 or 53 on the side of the element facing entry point 59 for the needle. In some cases, wings 55 rest against elements 51 or 53 to enable the elements to desirably resist movement of needle 14 in response to pressure from a patient's vascular system. A cut-out or slit in element 51 or 53, respectively, is not limited to any particular position or orientation on the element. For example, slit 52 or cut-out 54 can be located anywhere on the respective support element with respect to a longitudinal axis (not shown) or in any orientation with respect to the axis, for example, orthogonal to the axis. The following are not shown in the figures. It should be understood that respective support elements may include more than one slit 51 or cut-out 54 and that elements 51 or 53 may include both a slit and a cut-out. Respective multiple slits 52 on element 51 may have different respective depths 56. Respective multiple cut-outs 54 on element 53 may have different respective shapes, widths, and depths.

In some aspects (not shown), a plurality of slits and/or cut-outs are formed in element 51 or 53. Some or all of the slits and/or cut-outs have different respective depths. These different depths can be used to provide control of angle 18. In response to the different depths of the slits and/or cut-outs, angle 18 decreases or increases.

FIGS. 6A and 6B are perspective views of present invention device 60 with support element 62 movable with respect to fistula needle 14. The following should be viewed in light of FIGS. 6A and 6B. As noted supra, during the process of finding a suitable angle 18, the support element provides stable support for the fistula needle. In some aspects, element 62 is substantially non-resilient, element 62 is movable along surface 16, and angle 18 is controllable in response to the movement of element 62. For example, moving element 62 in direction 64 causes angle 18 to increase and moving element 62 in direction 66 causes angle 18 to decrease. Therefore, in FIG. 6A, angle 18 is larger than in FIG. 6B, since element 62 has been moved further in direction 64 in FIG. 6A. In FIGS. 6A and 6B, directions 64 and 66 are substantially parallel to fistula needle 14 and substantially orthogonal to a longitudinal axis (not shown) for element 62. However, it should be understood that element 62 can be moved in other directions and such movement is within the spirit and scope of the invention as claimed. In some aspects, element 62 also is resilient and as presented in the description for FIGS. 2A and 2B, angle 18 is controllable in response to pressure applied to element 62, for example, by pushing on needle 14. That is, the movement of element 62 can be performed alone or can be combined with a variation of pressure on element 62.

Returning to the general discussion of FIG. 1, element 12 can be formed in a variety of shapes. In some aspects, element 12 has a substantially longitudinal shape with respect to axis 19. That is, element 12 is generally longer in a direction orthogonal to an intended direction of use for fistula needle 14. In some aspects, element 12 has a uniform shape along axis 19. In some aspects (not shown), the shape of element 12 varies along axis 19. Element 12 is not limited to any particular cross-sectional shape or outline and can be made to have any number of such shapes or outlines. For example, shapes or outlines can be chosen to minimize production costs, to optimize a particular material, or to facilitate use with the fistula needle. Possible cross-sectional shapes or outlines include, but are not limited to: circular/round, ovoid, generally arcuate, three-sided, four-sided (e.g., square, rectangle, parallelogram or trapezoid), more than four sides (e.g., octagon), a combination of straight edges and arcuate edges, and amorphous. It should be understood that a same element 12 may have more than one cross-sectional outline.

In some aspects (not shown), element 12 has a composite construction. For example, a solid element 12 can have a core material with a specific resiliency or other characteristic and an outer portion with a different resiliency or other characteristic.

Element 12 is formed from a variety of materials. Some examples of these materials include, but are not limited to: cotton, in particular cotton rolls; open cell sponges, silicon sponges; natural and synthetic woven materials; rubber; and plastic. One applicable foam material is “Gold 33 polyurethane” produced by Avail Medical Corporation. Another applicable foam is a polyester reticulated Q or Q 100, produced by Foamex Corporation. One particular silicon material is “MC 300” produced by Rogers Corporation. However, it should be understood that element 12 can be formed of any material known in the art. It should be understood that the preceding discussion regarding element 12 is applicable to other aspects of the present invention.

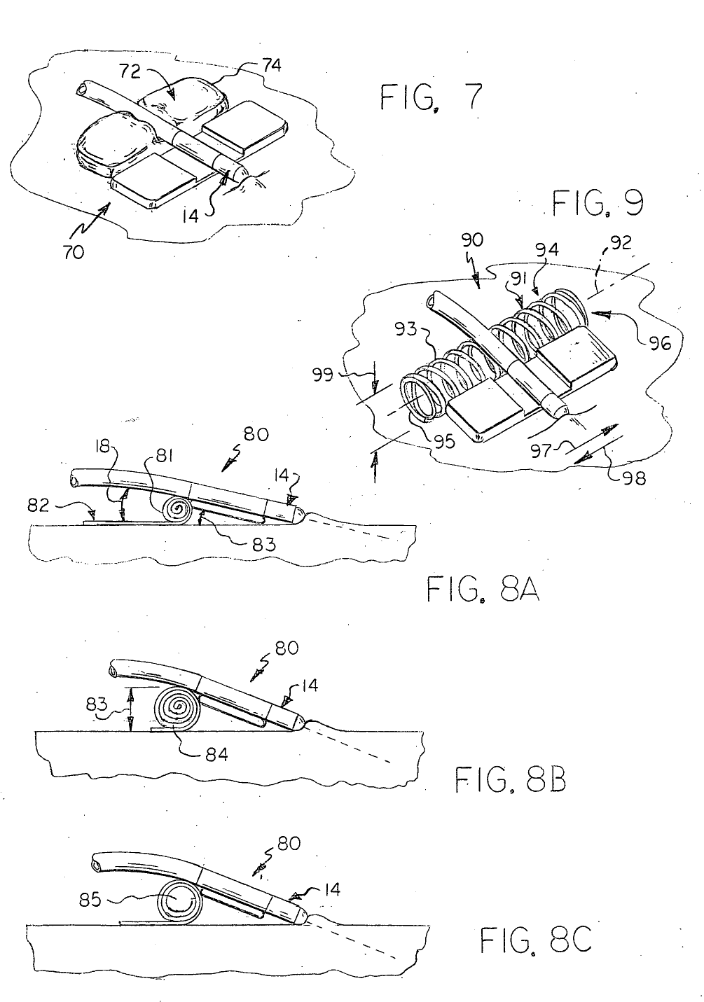

FIG. 7 is a perspective view of present invention device 70 with pouch support element 72. By pouch, we mean a configuration of a cover or outside component forming an internal space. For example, element 72 includes cover 74. In FIG. 7, element 72 is used to support fistula needle 14, shown in use with a patient's arm 16.

Element 72 can be filled with a variety of materials or fillings (not shown), for example, one or more gases, a solid mass, a plurality of pieces of a solid material, a liquid, and a combination of gases, liquids, or at least one piece of a solid material. By solid mass we mean a single piece of solid material. In some aspects, the one or more gases are atmospheric gases. In some aspects, the liquid is water. In some aspects, the liquid is substantially viscous. For example, the liquid has a higher viscosity that water. A more viscous liquid can be used to increase the “compression memory” and resiliency of element 72. In some aspects, the piece or pieces of materials are bead(s). The beads can be spherical or other shapes and can be of uniform or nonuniform sizes. For those aspects including liquids or solid materials, it should be understood that gases may also be included. For example, gases can be used to fill the spaces between beads. Cover 74 can be made of any material known in the art, including, but not limited to: natural and synthetic woven materials, plastic, and rubber.

FIGS. 8A, 8B, and 8C are side views of present invention device 80 with layered support element 81. The following should be viewed in light of FIGS. 8A-8C. Layered support element 81 is formed from a piece of material 82. In general, material 82 is substantially flat, but other configurations are possible. Material 82 is layered to form element 81 with a thickness 83 and angle 18 is controllable via the thickness. At least portions of material 82 are detachably connectable one to the other. By this we mean that the aforementioned portions adhere to each other without the use of outside force or other components or fasteners such as tape. Thus, the layering of element 81 is self-maintaining. In some aspects (not shown), material 82 has a hook and loop configuration or is at least partially coated with an adhesive, in particular, a pressure-sensitive adhesive.

In FIGS. 8A and 8B, material 82 is rolled to form element 81. In this case, thickness 83 is substantially a diameter of the rolled portion of element 81. In some aspects (not shown), material 82 is folded to form element 81. The manner in which material 82 is rolled can be varied to obtain various desired effects or configurations. For example, in FIGS. 8A and 8B, material 82 is ‘tightly’ rolled. That is, there is minimal space between layers 84 in element 81. In FIG. 8C, material 82 is more loosely rolled to form a substantially tubular shape with an opening 85.

Angle 18 is responsive to the layering of material 82 and is controllable through the layering. The layering of element 81 can be varied by a person using element 81. For example, the person may increase or decrease the rolling or folding as desired to obtain a desired angle 18. For example, the amount of material rolled in FIG. 8B is greater than the amount of material rolled in FIG. 8A. Therefore, height 83 is larger in FIG. 8B than in FIG. 8A. As a result, angle 18 is larger in FIG. 8B.

FIG. 9 is a perspective view of present invention device 90 with coil support element 91. Element 91 includes an axis 92 and in some aspects, element 91 is arranged such that fistula needle 14 is supported on element 91 substantially orthogonal to axis 92. In FIG. 9, the fistula needle is supported by surfaces 93 of coil 91. In some aspects (not shown), the fistula needle is arranged in spaces 94 between coils 95. In this case, angle 18 is adjustable by stretching or compressing element 91 along axis 92. For example, as end 96 is pulled in direction 97, spaces 94 become larger (coils 95 spread apart) and the fistula needle moves deeper into the coil structure of element 91, thereby decreasing angle 18. As end 96 is pushed in direction 98, spaces 94 become smaller (coils 95 move together) and the fistula needle is ‘pushed out’ of the coil structure of element 91, thereby increasing angle 18. In some aspects, height 99 is responsive to the movement of element 91 in directions 97 and 98. For example, height 99 increases as end 96 is pushed in direction 98. When the needle rests on surfaces 93, the change in height 99 results in a change of angle 18. For example, increasing height 99 increases angle 18. In some aspects, directions 97 and 98 are parallel to axis 92, however, other orientations with respect to axis 92 are possible. It should be understood that any combination of stretching, compressing, or moving element 90 is included in the spirit and scope of the invention as claimed.

In some aspects (not shown), the shape of present invention devices is further varied. For example, returning to FIGS. 2A and 2B, element 22 can be varied along a longitudinal axis (not shown) for the element such that moving element 22 in a direction parallel to the axis varies angle 18. For example, element 22 can be formed such that the height of element 22 with respect to arm 16 decreases uniformly from end 100 to end 102. In this case, as element 12 moves in direction 104 and fistula needle 14 is held in a same position with respect to direction 104, angle 18 increases. As element 22 moves in direction 106 and fistula needle 14 is held in a same position with respect to direction 106, angle 18 decreases. Directions 104 and 106 are substantially orthogonal to fistula needle 14 and substantially parallel to the axis noted supra. However, it should be understood that other orientations of directions 104 and 106 with respect to the fistula needle and the axis are possible. It also should be understood that changes in height can be linear, non-linear, or can increase and decrease for a same element 22. It should be understood that the above discussion is applicable to other embodiments of the present invention.

FIG. 10 is a front view of present invention device 110 with support element 112 having shaped bottom surface 114. In accordance with the physical characteristics and configuration of a present invention support element, the element may tend to conform to the surface upon which the element is resting in response to pressure applied to the element. For example, returning to FIG. 1, as element 12 is taped to a patient's arm, element 12 may tend to curve or otherwise adapt to arm 16. However, in some aspects, to accentuate the adaptation of element 112 to surface 116, bottom surface 114 is shaped to complement surface 116. Typically, surface 116 is part of the body of a patient upon whom a fistula needle is being used. For example, in FIG. 10, surface 116 is part of arm 16 shown in FIG. 1. Surface 114 creates greater comfort for the person upon whom element 112 is placed and can prevent undesired motion of element 112. Surface 114 can be configured in any shape or combination of shapes. In some aspects, the resiliency and other characteristics of the material or materials forming element 112 are considered in configuring surface 114.

The following should be viewed in light of FIGS. 1-10. It should be understood that the various characteristics of the present invention aspects shown in FIGS. 1 through 10 can be combined. For example, elements 42, 44, 51, 53, 62, 81, 91, and 112 can be resilient. Then the discussion regarding resiliency in the description of FIGS. 2A and 2B is applicable. Also, elements 22, 32, 42, 44, 51, 53, 72, 81, 91, and 112 can be made moveable with respect to the needle. Then the discussion in the description of FIGS. 6A and 6B is applicable. Elements 22, 42, 44, 51, 53, 62, 112 can be made to include a void, for example, to be tubular. Then the discussion in the description of FIG. 3 is applicable. Resiliency, movement, tubular shape, and any other attributes described for the present invention can be combined.

As an option, present invention devices can be processed to make the devices sterile or to at least have a very high kill rate for target microorganisms. However, it should be understood that the present invention is not limited to sterile applications or to applications involving very high kill rate for target microorganisms.

In some aspects (not shown), device 10 is used to support other medical devices or instruments. For example, for non-invasive surgical procedures, device 10 can be used to support instruments involved in the surgery. Specifically, device 10 can be used to control an angle of entry for the instruments. For example, device 10 can be used to support a fiber optic line for a miniature camera or lighting apparatus.

Thus, it is seen that the objects of the invention are efficiently obtained, although changes and modifications to the invention should be readily apparent to those having ordinary skill in the art, without departing from the spirit or scope of the invention as claimed. Although the invention is described by reference to a specific preferred embodiment, it is clear that variations can be made without departing from the scope or spirit of the invention as claimed.

What is claimed is:

1. A device for controlling an angle of cannulation for a fistula needle, comprising:
a pre-formed support element operatively arranged to support said fistula needle and manipulatable to control said angle of cannulation.

2. The device recited in claim 1 wherein said support element is resilient and said support element is arranged to control said angle in response to pressure upon said support element.

3. The device recited in claim 1 wherein said support element comprises a solid shape.

4. The device recited in claim 1 wherein said support element comprises at least one void.

5. The device recited in claim 1 wherein said support element comprises a cross-section with an outline and said outline is selected from the group consisting of round, ovoid, arcuate, three-sided, four-sided, having more than four sides, having straight edges and arcuate edges, and amorphous.

6. The device recited in claim 1 wherein said support element is a pouch.

7. The device recited in claim 6 wherein said pouch is at least partially filled with a material selected from the group consisting of one or more first gases; a solid mass; a plurality of pieces of a first solid material; a first liquid; and a combination of one or more second gases, or a second liquid, or at least one piece of a second solid material.

8. The device recited in claim 1 wherein said support element comprises a coil.

9. The device recited in claim 1 wherein said support element is configured to holdingly engage said fistula needle.

10. The device recited in claim 9 wherein said support element comprises a slit arranged to receive said fistula needle or a cut-out arranged to receive said fistula needle.

11. The device recited in claim 1 wherein said angle is with respect to a surface and wherein said manipulatable to control said angle of cannulation further comprises said support element being movable along said surface.

12. A device for controlling an angle of cannulation for a fistula needle, comprising:
a piece of material layered to form a thickness and operatively arranged to support said fistula needle, wherein at least portions of said material are detachably connectable to each other and said angle is controllable via said thickness.

13. The device recited in claim 12 wherein said piece of material is folded.

14. The device recited in claim 12 wherein said piece of material is rolled.

15. The device recited in claim 12 wherein said layered piece of material comprises at least one void.

16. The device recited in claim 12 wherein said layered material is resilient and said layered material is arranged to control said angle in response to pressure upon said layered material.

17. The device recited in claim 12 wherein said angle is with respect to a surface and wherein said support element is operatively arranged to move along said surface and to control said angle in response to said movement.

18. A combination for controlling an angle of cannulation, comprising:
a pre-formed support element; and,
a fistula needle, wherein said support element is arranged to support said fistula needle and to move with respect to said fistula needle and wherein an angle of cannulation for said fistula needle is controllable via said movement of said support element.

19. The combination recited in claim 18 wherein said support element is resilient and said support element is arranged to control said angle is response to pressure upon said support element.

20. The combination recited in claim 18 wherein said support element is a pouch.

21. The combination recited in claim 18 wherein said support element comprises a piece of material layered to form a thickness, at least portions of said material are detachably connectable to each other, and said angle is controllable via said thickness.

22. The combination recited in claim 21 wherein said piece of material is folded or rolled.

23. The combination recited in claim 18 wherein said support element comprises a coil.

24. The combination recited in claim 18 wherein said support element is configured to holdingly engage said fistula needle.

25. A method for controlling an angle of cannulation for a fistula needle, comprising:
supporting said fistula needle on a preformed support element; and,
manipulating said support element to control said angle.

26. The method of claim 25 wherein said support element is resilient and manipulating said support element further comprises applying pressure to said support element.

27. The method of claim 25 wherein said support element comprises a pouch.

28. The method of claim 25 wherein said support element comprises a coil.

29. The method of claim 25 further comprising:
configuring said support element to holdingly engage said fistula needle.

30. The method of claim 25 wherein said angle is with respect to a surface and wherein manipulating said support element further comprises moving said support element along said surface.

31. A method for controlling an angle of cannulation for a fistula needle, comprising:
layering a piece of material to form a thickness, wherein at least portions of said material are detachably connectable to each other;
supporting said fistula needle with said layered material; and,
controlling said angle via said thickness.

32. The method of claim 31 wherein layering further comprises folding or rolling.

33. The method of claim 31 wherein said layered material is resilient; and,
said method further comprising:
applying pressure to said support element; and,
controlling said angle responsive to said applying pressure.

34. The method of claim 31 wherein said angle is with respect to a surface; and,
said method further comprising:
moving said support element along said surface; and,
controlling said angle responsive to said movement.

35. A method for controlling an angle of cannulation for a fistula needle, comprising:
supporting said fistula needle on a pre-formed support element;
moving said support element with respect to said fistula needle; and,
controlling said angle responsive to said movement of said support element.

36. The method of claim 35 wherein said support element is resilient; and,
said method further comprising:
applying pressure to said support element; and,
controlling said angle responsive to said applying.

37. The method of claim 35 wherein said support element is a pouch.

38. The method of claim 35 wherein said support element comprises a piece of material, where at least portions of said material are detachably connectable to each other; and,
said method further comprising:
layering said piece of material to form a thickness; and,
controlling said angle responsive to said thickness.

39. The method of claim 38 wherein said layering further comprises folding or rolling.

40. The method of claim 35 wherein said support element comprises a coil.

41. The method of claim 35 further comprising:
configuring said support element to holdingly engage said fistula needle.
